(19) United States
(12) Patent Application Publication
(10) Pub. No.: US 2013/0237590 A1
(43) Pub. Date: Sep. 12, 2013

(54) ASSESSING THYROID NEOPLASMS

(71) Applicants: Norman L. Eberhardt, Rochester, MN (US); Bryan Mciver, Rochester, MN (US); Stefan K.G. Grebe, Rochester, MN (US); Honey V. Reddi, Rochester, MN (US); Alicia Algeciras-Schimnich, Rochester, MN (US)

(72) Inventors: Norman L. Eberhardt, Rochester, MN (US); Bryan Mciver, Rochester, MN (US); Stefan K.G. Grebe, Rochester, MN (US); Honey V. Reddi, Rochester, MN (US); Alicia Algeciras-Schimnich, Rochester, MN (US)

(73) Assignee: MAYO FOUNDATION FOR MEDICAL EDUCATION AND RESEARCH, Rochester, MN (US)

(21) Appl. No.: 13/810,363

(22) PCT Filed: Jul. 14, 2011
(86) PCT No.: PCT/US2011/043943

§ 371 Date: May 28, 2013

Related U.S. Application Data
(60) Provisional application No. 61/364,717, filed on Jul. 15, 2010.

(51) Int. Cl. A61K 31/7088

(52) U.S. Cl. CPC A61K 31/7088
USPC 514/44 R

(57) ABSTRACT

This document provides methods and materials involved in assessing thyroid neoplasms. For example, methods and materials for using microRNA (miR) levels (e.g., miR122 levels) to determine whether a thyroid neoplasm (e.g., a follicular thyroid neoplasm) is benign or malignant as well as methods and materials for using miR levels (e.g., miR122 levels) to determine whether a malignant thyroid cancer patient is likely to have a favorable or unfavorable outcome are provided. This document also provides methods and materials involved in treating cancer.

### CROSS-REFERENCE TO RELATED APPLICATIONS

This application claims the benefit of U.S. Provisional Application Ser. No. 61/364,717, filed Jul. 15, 2010. The disclosure of the prior application is considered part of (and is incorporated by reference in) the disclosure of this application.

### STATEMENT AS TO FEDERALLY SPONSORED RESEARCH

This invention was made with government support under grant CA080117 awarded by the National Institutes of Health's National Cancer Institute. The government has certain rights in the invention.

### BACKGROUND

1. Technical Field

This document relates to methods and materials involved in assessing thyroid neoplasms. For example, this document relates to methods and materials for using microRNA (miR) levels (e.g., miR122 levels) to determine whether a thyroid neoplasm (e.g., a follicular thyroid neoplasm) is benign or malignant as well as methods and materials for using miR levels (e.g., miR122 levels) to determine whether a malignant thyroid cancer patient is likely to have a favorable or unfavorable outcome. This document also relates to methods and materials involved in treating cancer (e.g., cancers lacking PPFP polypeptide expression).

2. Background Information

Follicular thyroid carcinoma (FTC) accounts for about 20 percent of all thyroid cancers and up to 40 percent of the deaths associated with this disease. In iodine-deficient areas, its incidence can be twice as high. Unlike most well-differentiated thyroid cancers, which are readily treated and yield excellent outcomes, FTC, when diagnosed at a late stage, can be very resistant to treatment, with 10-yr survival rates less than 40 percent.

### SUMMARY

This document provides methods and materials related to assessing thyroid neoplasms. For example, this document provides methods and materials for using miR levels (e.g., miR122 levels) to determine whether a thyroid neoplasm (e.g., a follicular thyroid neoplasm) is benign or malignant and methods and materials for using miR levels (e.g., miR122 levels) to determine whether a malignant thyroid cancer patient is likely to have a favorable or unfavorable outcome. Determining if a mammal (e.g., a human patient) has either a benign or malignant thyroid neoplasm can allow physicians and the patient, in the case of humans, to determine a course of treatment or monitoring appropriate for that patient. For example, a patient found to have a malignant thyroid neoplasm can elect to undergo appropriate anti-cancer treatments such as surgery (lobectomy, thyroidectomy, or lymphadenectomy), radioactive iodine treatment, external radiation therapy, or chemotherapy treatment. Likewise, determining if a mammal (e.g., a human patient) has a likelihood for a favorable or unfavorable outcome can allow physicians and the patient, in the case of humans, to determine a course of treatment or monitoring appropriate for that patient. For example, a patient found to have a likelihood for a favorable outcome may elect to forgo experimental treatments that may cause significant toxicity, while a patient found to have a likelihood for an unfavorable outcome may elect to go forward with such experimental treatments.

In general, one aspect of this document features a method for determining whether a mammal has a benign or malignant follicular thyroid neoplasm. The method comprises, or consists essentially of, (a) determining whether or not cells of the neoplasm comprise an elevated level of miR122, (b) classifying the mammal as having a benign follicular thyroid neoplasm if the cells lack the elevated level of the miR122, and (c) classifying the mammal as having malignant follicular thyroid neoplasm if the cells have the elevated level of the miR122. The mammal can be a human. The elevated level can be a level greater than the level of miR122 present within normal thyrocytes. The elevated level can be a level greater than the level of miR122 present within normal thyroid cells. The elevated level can be a level at least two times greater than the level of miR122 present within normal thyroid cells. The elevated level can be a level at least three times greater than the level of miR122 present within normal thyroid cells. The elevated level can be a level at least six times greater than the level of miR122 present within normal thyroid cells. The cells can be cells of a fine-needle aspiration biopsy of the neoplasm.

Another aspect of this document features a method for identifying a mammal as having a benign follicular thyroid neoplasm. The method comprises, or consists essentially of, (a) detecting the absence of cells of the neoplasm having an elevated level of miR122, and (b) classifying the mammal as having the benign follicular thyroid neoplasm. The mammal can be a human. The elevated level can be a level greater than the level of miR122 present within normal thyroid cells. The elevated level can be a level that is about two times greater than the level of miR122 present within normal thyroid cells. The cells can be cells of a fine-needle aspiration biopsy of the neoplasm.

Another aspect of this document features a method for identifying a mammal as having a malignant follicular thyroid neoplasm. The method comprises, or consists essentially of, (a) detecting the presence of cells of the neoplasm having an elevated level of miR122, and (b) classifying the mammal as having the malignant follicular thyroid neoplasm. The mammal can be a human. The elevated level can be a level greater than the level of miR122 present within normal thyroid cells. The elevated level can be a level at least two times greater than the level of miR122 present within normal thyroid cells. The elevated level can be a level at least three times greater than the level of miR122 present within normal thyroid cells. The elevated level can be a level at least six times greater than the level of miR122 present within normal thyroid cells. The cells can be cells of a fine-needle aspiration biopsy of the neoplasm.

Another aspect of this document features a method for determining the likelihood that a malignant follicular thyroid neoplasm patient will have a favorable outcome, wherein the method comprises, or consists essentially of, (a) determining whether or not cells of a neoplasm of the patient have an elevated level of miR122 as compared to the average level of miR122 present in follicular thyroid carcinoma cells that lack expression of a PAX8/PPARγ fusion protein (PPFP), (b) classifying the patient as having a likelihood of having an unfavorable outcome if the cells lack the elevated level of the miR122, and (c) classifying the patient as having a likelihood of having a favorable outcome if the cells have the elevated level of the miR122. The patient can be a human. The cells can be cells of a fine-needle aspiration biopsy of the neoplasm.

Another aspect of this document features a method for identifying a malignant follicular thyroid neoplasm patient as having a likelihood for a favorable outcome, wherein the method comprises, or consists essentially of, (a) detecting the presence of cells of a neoplasm of the patient having an elevated level of miR122 as compared to the average level of miR122 present in follicular thyroid carcinoma cells that lack expression of a PPFP, and (b) classifying the mammal as having the likelihood for a favorable outcome. The mammal can be a human. The cells can be cells of a fine-needle aspiration biopsy of the neoplasm.

Another aspect of this document features a method for identifying a malignant follicular thyroid neoplasm patient as having a likelihood for an unfavorable outcome, wherein the method comprises, or consists essentially of, (a) detecting the absence of cells of a neoplasm of the patient having an elevated level of miR122 as compared to the average level of miR122 present in follicular thyroid carcinoma cells that lack expression of a PPFP, and (b) classifying the mammal as having the likelihood for an unfavorable outcome. The mammal can be a human. The cells can be cells of a fine-needle aspiration biopsy of the neoplasm.

Another aspect of this document features a method for treating a mammal having cancer cells that lack expression of a PPFP polypeptide. The method comprises, or consists essentially of, administering, to the mammal, a nucleic acid comprising a nucleic acid sequence that encodes a PPFP polypeptide under conditions wherein the cancer cells express the PPFP polypeptide. The mammal can be a human. The cancer cells can be colon, melanoma, follicular thyroid cancer, or anaplastic thyroid cancer cells. The nucleic acid can be administered to the mammal under conditions wherein expression of microRNA 122 (miR122) in the cancer cells is increased. The volume of a tumor comprising the cancer cells can decrease after the administration. The decrease can be a decrease of at least about 10 percent. The decrease can be a decrease of at least about 25 percent.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a graph plotting the fold increase in miR122 expression in 6 fresh frozen FTC samples (FTC(+PPFP)) containing a PAX8/PPARγ rearrangement that results in expression of a PAX8/PPARγ fusion protein (PPFP) as compared to miR122 expression in 6 fresh frozen FTC samples (FTC(−PPFP)) lacking PPFP expression. FIG. 1B is a graph plotting the fold increase in miR122 expression in 4 formalin fixed paraffin embedded FTC samples with PPFP expression (FTC(+PPFP)) as compared to miR122 expression in 6 formalin fixed paraffin embedded FTC samples lacking PPFP expression (FTC(−PPFP)). FIG. 1C is a graph plotting the fold increase in miR-122 expression in 7 benign follicular adenoma (FA) tissue samples versus 7 normal thyroid tissue samples. FIG. 1D is a graph comparing the fold increase in miR-122 expression in an adenoma model consisting of immortalized thyrocytes (Nthy-ori 3-1 [NT] cells): NT-Vector and NT-PPFP cells in vitro versus tumor xenografts in vivo. Asterisks indicate statistical significance at the p<0.05 level. Error bars indicate SEM.

FIG. 2A contains a graph plotting the miR122 expression for the indicated cells. FIG. 2B is a graph plotting absorbance at 490 nm versus time, indicating cell growth of the indicated cells. FIG. 2C is a graph plotting tumor volume post injection for mice injected with the indicated cells.

FIG. 3A is a schematic of the PPFP expression construct. FIG. 3B contains a photograph of a Western blot demonstrating the expression of PTEN in FTC-derived cell lines. FIG. 3C contains a photograph of an ethidium bromide stained agarose gel with the RT-PCR product, demonstrating the expression of PPFP RNA in FTC-derived cell lines. FIG. 3D is a graph plotting absorbance at 490 nm versus time, indicating cell growth of the indicated cells. FIGS. 3E and 3F are graphs plotting tumor volume post injection for mice injected with the indicated cells. Images of tumors shown were taken at the time of harvest, 7 and 4 weeks, for WRO and FTC-133 cells, respectively. FIGS. 3G and 3H contain graphs plotting the miR122 expression for the indicated cells. FIG. 3I is a graph that includes data from FIG. 3G and plots fold increase in miR-122 expression in WRO cells and xenografts expressing either vector or constitutively expressed PPFP.

FIG. 4A is a schematic of a dominant-negative PPARγ (DN-PPARγ) expression construct. FIG. 4B is a photograph of a Western blot of PPARγ (top panel), DN-PPARγ (middle panel), and β-actin (bottom panel) in WRO stable cells transfected with vector, PPFP, or DN-PPARγ-Flag. FIG. 4C is a graph plotting the endogenous PPARγ activity for the indicated cells. FIG. 4E is a graph plotting absorbance at 490 nm versus time, indicating cell growth of the indicated cells. FIG. 4E is a graph plotting tumor volume post injection for mice injected with the indicated cells (*p<0.05, **p<0.005). FIG. 4F is a graph plotting the miR122 expression for the indicated cells. FIG. 4G is a graph plotting PPARγ activity in the presence of GW9662, a PPARγ antagonist. FIG. 4H is a graph plotting the miR122 expression in the presence and absence of GW9662-treated WRO-vector cells. FIG. 41 is a graph plotting PPARγ activity through its PPARγ response element (PPRE) in the indicated cells.

FIGS. 5A and B contain a photograph (FIG. 5A) of a Western blot of PTEN (top panel) and β-actin (bottom panel) after transient ectopic expression of PTEN and a graph (FIG. 5B) plotting the miR122 expression levels in FTC-133-Vector or PPFP cells transiently expressing pcDNA3.1-PTEN.

FIG. 6A is a graph plotting the ratio of the expression of ADAM-17 mRNA to miR-122 expression for the indicated cells. FIG. 6B is a graph plotting the ratio of the expression of ADAM-17 mRNA to miR-122 expression in FTC with and without PPFP. FIG. 6C is a graph plotting the ratio of the expression of ADAM-17 mRNA to miR-122 expression or normal tissue samples compared to benign follicular adenoma (FA) tissue samples.

FIG. 7A contains photographs of immunohistochemical staining of WRO xenograft tumors of CD-31 for the quantitation of microvessel density. FIG. 7B is graph plotting the percent CD-31 staining intensity.

FIG. 8A contains a photograph of an ethidium bromide stained agarose gel with the RT-PCR product, demonstrating the expression of PPFP in a colon cancer cell line, ARO. FIG. 8B contains a graph plotting the tumor volume for mice injected with ARO cells with PPFP expression and without PPFP expression (Vector_GFP).

FIG. 9A contains a photograph of an ethidium bromide stained agarose gel with the RT-PCR product, demonstrating the expression of PPFP in a melanoma cell line, DRO. FIG. 9B contains a graph plotting the tumor volume for mice injected with DRO cells with PPFP expression and without PPFP expression (Vector_GFP).

FIG. 10A contains a photograph of an ethidium bromide stained agarose gel with the RT-PCR product, demonstrating the expression of PPFP in an anaplastic thyroid cancer cell line, FRO. FIG. 10B contains a graph plotting the tumor volume for mice injected with FRO cells with PPFP expression and without PPFP expression (Vector_GFP). FIG. 10C contains a photograph of a Western blot demonstrating the expression of PPFP in an anaplastic thyroid cancer cell line, KTC-3. FIG. 10D contains a graph plotting the tumor volume for mice injected with KTC-3 cells with PPFP expression and without PPFP expression (Vector_GFP).

FIG. 11 contains a nucleic acid sequence (SEQ ID NO:4) that encodes a PPFP polypeptide and an amino acid sequence (SEQ ID NO:3) of a PPFP polypeptide. The pax8 sequence of FIG. 11 can be replaced with splice variants of pax8 such as those described elsewhere (Placzkowski et al., PPAR Research, Vol. 2008, Article ID 672829, 10 pgs. (2008); Kroll et al., Science, 289(5483):1357-1360 (2000); Marques et al., J. Clin. Endocrin. Metabol., 87(8):3947-3952 (2002); and Cheung et al., J. Clin. Endocrin. Metabol., 88(1):354-357 (2003)).

### DETAILED DESCRIPTION

This document provides methods and materials related to assessing thyroid neoplasms. For example, this document provides methods and materials for using miR levels (e.g., miR122 levels) to determine whether a thyroid neoplasm (e.g., a follicular thyroid neoplasm) is benign or malignant. This document also provides methods and materials for using miR levels (e.g., miR122 levels) to determine whether a malignant thyroid cancer patient is likely to have a favorable or unfavorable outcome. As disclosed herein, the detection of an elevated level of miR122 in thyroid neoplastic cells as compared to the miR122 level present in normal thyroid cells can indicate that the patient's thyroid neoplasm is malignant. In addition, the detection of an elevated level of miR122 in malignant thyroid neoplastic cells as compared to the miR122 level present in malignant thyroid neoplastic cells lacking PPFP expression can indicate that the patient has a likelihood for a favorable outcome. The nucleic acid sequence of miR122 is set forth in GenBank GI no. 262205241 (accession No. NR_{—}029667.1).

Any appropriate method can be used to determine the level of miR122 present within cells of a mammal (e.g., a human, dog, cat, horse, or monkey). For example, RT-PCR, quantitative PCR, and RNase protection assay techniques can be used to assess miR122 levels. Any appropriate sample can be obtained and assessed for miR122. For example, fine-needle aspiration biopsies or surgical biopsies of a thyroid neoplasm or blood samples (e.g., serum or plasma samples) can be obtained, and the level of miR122 expression within the cells of such samples can be determined as described herein.

The term “elevated level” as used herein with respect to the level of miR122 can be in comparison with the median miR122 level present in normal thyroid cells (e.g., the median miR122 level determined from a random sampling of 5, 10, 15, 20, 30, 40, 50, 100, 500, or more thyroid samples from humans known not to have a thyroid disorder such as thyroid cancer). In such cases, the presence of an elevated level can indicate that the patient's thyroid neoplasm is malignant, while the absence of such an elevated level can indicate that the patient's thyroid neoplasm is benign.

The term “elevated level” as used herein with respect to the level of miR122 can be in comparison with the median miR122 level present in malignant thyroid neoplastic cells lacking PPFP expression. In such cases, the presence of an elevated level can indicate that the malignant thyroid cancer patient has a likelihood for a favorable outcome, while the absence of such an elevated level can indicate that the malignant thyroid cancer patient has a likelihood for an unfavorable outcome.

This document also provides methods and materials to assist medical or research professionals in determining whether a thyroid neoplasm (e.g., a follicular thyroid neoplasm) is benign or malignant and methods and materials to assist medical or research professionals in determining whether a malignant thyroid cancer patient is likely to have a favorable or unfavorable outcome. Medical professionals can be, for example, doctors, nurses, medical laboratory technologists, and pharmacists. Research professionals can be, for example, principal investigators, research technicians, postdoctoral trainees, and graduate students. A professional can be assisted by (1) determining the level of miR122 as described herein, and (2) communicating information about the level to that professional.

Any appropriate method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document also provides methods and materials related to treating mammals (e.g., humans) having cancer (e.g., colon cancer, melanoma, anaplastic thyroid cancer, or follicular thyroid cancer). Any appropriate method can be used to identify a mammal as having cancer. For example, standard melanoma biopsy techniques can be used to identify humans having melanoma. In some cases, the methods and materials provided herein can be used to treat a mammal having cancer cells that lack expression of a PPFP polypeptide. As described herein, a mammal identified as having cancer (e.g., a cancer lacking expression of a PPFP polypeptide) can be treated by administering a nucleic acid encoding a PPFP polypeptide to the mammal such that the level of PPFP polypeptide expression and/or the level of miR122 is increased.

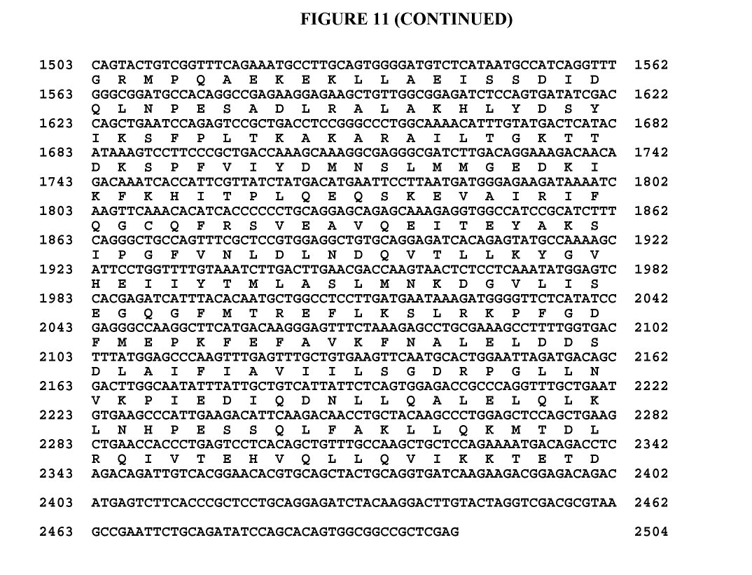

As described herein, the level of PPFP polypeptide expression can be increased in a mammal having cancer by administering a nucleic acid encoding a PPFP polypeptide to the mammal. Such a nucleic acid can encode a full-length PPFP polypeptide such as a human PPFP polypeptide having the amino acid sequence set forth in FIG. 11. In some cases, a PPFP polypeptide can have the pax8 sequence of FIG. 11 replaced with a splice variant of pax8 such as a pax8 splice variant described elsewhere (Placzkowski et al., PPAR Research, Vol. 2008, Article ID 672829, 10 pgs. (2008); Kroll et al., Science, 289(5483):1357-1360 (2000); Marques et al., J. Clin. Endocrin. Metabol., 87(8):3947-3952 (2002); and Cheung et al., J. Clin. Endocrin. Metabol., 88(1):354-357 (2003)). A nucleic acid encoding a PPFP polypeptide can be administered to a mammal using any appropriate method. For example, a nucleic acid can be administered to a mammal using a vector such as a viral vector.

Vectors for administering nucleic acids (e.g., a nucleic acid encoding a PPFP polypeptide) to a mammal are known in the art and can be prepared using standard materials (e.g., packaging cell lines, helper viruses, and vector constructs). See, for example, Gene Therapy Protocols (Methods in Molecular Medicine), edited by Jeffrey R. Morgan, Humana Press, Totowa, N.J. (2002) and Viral Vectors for Gene Therapy: Methods and Protocols, edited by Curtis A. Machida, Humana Press, Totowa, N.J. (2003). Virus-based nucleic acid delivery vectors are typically derived from animal viruses, such as adenoviruses, adeno-associated viruses, retroviruses, lentiviruses, vaccinia viruses, herpes viruses, and papilloma viruses.

Vectors for nucleic acid delivery can be genetically modified such that the pathogenicity of the virus is altered or removed. The genome of a virus can be modified to increase infectivity and/or to accommodate packaging of a nucleic acid, such as a nucleic acid encoding a PPFP polypeptide. A viral vector can be replication-competent or replication-defective, and can contain fewer viral genes than a corresponding wild-type virus or no viral genes at all.

In addition to nucleic acid encoding a PPFP polypeptide, a viral vector can contain regulatory elements operably linked to a nucleic acid encoding a PPFP polypeptide. Such regulatory elements can include promoter sequences, enhancer sequences, response elements, signal peptides, internal ribosome entry sequences, polyadenylation signals, terminators, or inducible elements that modulate expression (e.g., transcription or translation) of a nucleic acid. The choice of element(s) that may be included in a viral vector depends on several factors, including, without limitation, inducibility, targeting, and the level of expression desired. For example, a promoter can be included in a viral vector to facilitate transcription of a nucleic acid encoding a PPFP polypeptide. A promoter can be constitutive or inducible (e.g., in the presence of tetracycline), and can affect the expression of a nucleic acid encoding a PPFP polypeptide in a general or tissue-specific manner.

As used herein, “operably linked” refers to positioning of a regulatory element in a vector relative to a nucleic acid in such a way as to permit or facilitate expression of the encoded polypeptide. For example, a viral vector can contain a neuronal-specific enolase promoter and a nucleic acid encoding a PPFP polypeptide. In this case, the enolase promoter is operably linked to a nucleic acid encoding a PPFP polypeptide such that it drives transcription in neuronal cancers.

A nucleic acid encoding a PPFP polypeptide also can be administered to a mammal using non-viral vectors. Methods of using non-viral vectors for nucleic acid delivery are known to those of ordinary skill in the art. See, for example, Gene Therapy Protocols (Methods in Molecular Medicine), edited by Jeffrey R. Morgan, Humana Press, Totowa, N.J. (2002). For example, a nucleic acid encoding a PPFP polypeptide can be administered to a mammal by direct injection of nucleic acid molecules (e.g., plasmids) comprising nucleic acid encoding a PPFP polypeptide, or by administering nucleic acid molecules complexed with lipids, polymers, or nanospheres.

A nucleic acid encoding a PPFP polypeptide can be produced by standard techniques, including, without limitation, common molecular cloning, polymerase chain reaction (PCR), chemical nucleic acid synthesis techniques, and combinations of such techniques. For example PCR or RT-PCR can be used with oligonucleotide primers designed to amplify nucleic acid (e.g., genomic DNA or RNA) encoding a PPFP polypeptide. Once obtained, a nucleic acid encoding a PPFP polypeptide can then be used to generate a viral vector, for example, which can be administered to a mammal so that the level of a PPFP polypeptide and/or the level of miR122 within cancer cells of the mammal is increased.

The invention will be further described in the following examples, which do not limit the scope of the invention described in the claims.

### EXAMPLES

### Example 1

### PAX8/PPARγ Fusion Protein Modulates Follicular Thyroid Tumorigenesis in Part via PTEN Mediated Up-Regulation of Tumor Suppressor miR-122

### Generation of Stable Cells

WRO cells (obtained from Dr. John A Copland, Mayo Clinic Jacksonville) were grown in RPMI-1640 medium (Invitrogen, Carlsbad, Calif.) supplemented with 10% FBS (Hyclone, Logan, Utah) and 1× penicillin-streptomycin (Invitrogen) at 37° C. in humidified conditions with 5% CO². FTC-133 cells (obtained from Dr. Matthew Ringel, The Ohio State University) were grown in DMEM with 10% FBS, 1× nonessential amino acids (1%) and Pen-strep. PPFP and dominant negative PPARγ constructs were generated as described elsewhere (Reddi et al., Expression of the PAX8/PPARγ Fusion Protein Is Associated with Decreased Neovascularization In Vivo: Impact on Tumorigenesis and Disease Prognosis, Genes and Cancer, (2010)). The PTEN expression plasmid was provided by Dr. Kausthub Datta, Mayo Clinic, Rochester, Minn.

To generate stable lines, cells were seeded at 1×10⁶ cells per 100 mm petri dish and transfected 24 hours later in multiples of six, with 10 μg each of vector or PPFP using LT-1 (Minus Bio, Madison, Wis.). For the generation of DN-PPARγ and miR stable cells, the DN-PPARγ construct (Reddi et al., Expression of the PAX8/PPARγ Fusion Protein Is Associated with Decreased Neovascularization In Vivo: Impact on Tumorigenesis and Disease Prognosis, Genes and Cancer, (2010)), mmu-Null, and mmu-miR122-EGFP constructs (Cellbio labs) were used. After 72 hours, WRO-Vector, WRO-PPFP, WRO-DN-PPARγ, FTC-Vector, and FTC−PPFP cells were obtained by selection in media containing 400 μg/mL of G418 (Invitrogen) with media changes every 72 hours. WRO-miR-Null and WRO-miR-122 cells were generated using 2.5 μg/mL puromycin selection. Pools of cells were harvested and analyzed for mRNA and microRNA expression by quantitative RT-PCR and for polypeptide expression by Western blotting.

RNA Extraction, RT-PCR for PPFP, miR-122

RNA from each group of cells was isolated using TriZol Reagent (Invitrogen). RNA (1 μg) was reverse transcribed with oligo-dT primers using Message Sensor RT kit (Ambion, Austin, Tex.). PAX8/PPARγ fusion gene was detected by PCR using 5 μL of cDNA and a sense primer from exon 7 of PAX8 (5′-CGCGGATCCGCATT-GACTCACAGAGCA-3; SEQ ID NO:1; see, also, GenBank Accession No. NM_{—}013953 and GI No.81295803) and antisense primer from exon 1 of PPARγ1 (5′-CCGGAATTCGAAGTCAACAGTAGTGAA-3; SEQ ID NO:2; GenBank Accession No. NM_{—}138712 and GI No. 116284369). Glyceraldehyde 3-phosphate dehydrogenase (GAPDH) was amplified using kit primers. Xenografts were harvested at weeks 1 and 2 post induction and homogenized in T-PER (Pierce, Rockford, Ill.). RNA was extracted, and RT-PCR was performed for PPFP and GAPDH. Annealing temperature for PPFP and GAPDH was 55° C. for 30 seconds. miR-122 status was verified using TaqMan microRNA assays (Applied Biosystems) as per the manufacturer's instructions.

### Luciferase Reporter Assays

Cells (0.3×10⁶ cells/well) in six well plates were transfected using LT-1 reagent (Mirus, Madison Wis.) with 10 ng of Renilla (Promega, Madison, Wis.) and 100 μg of PPRE-Aco-Luc reporter in triplicate. After 24 hours, cells were lysed and assayed using the dual luciferase kit (Promega). Data was calculated as a ratio of the luciferase to renilla expression and plotted as fold-change compared to vector. To test the effect of GW9662 on PPARγ activity, cells were treated 18 hours post transfection with either 2 μL DMSO or 10 μM GW9662 (a potent antagonist of PPARγ) for 24 hours prior to harvest and assay.

### MicroRNA Arrays

Global miRNA expression was assessed in 26 fresh frozen thyroid tissues including 8 FA (1 with and 7 without PPFP) and 12 FTC (6 with and 6 without PPFP) and 7 normal samples using the human v2 miR panel from Illumina Ratios were generated between different groups. Fold-changes were calculated, sorted with mean expression differences ≧1, 2, or 3 SD, and then analyzed in independent groups. The expression of miR-122 was confirmed in the same set of tumors using quantitative RT-PCR and also validated in another independent cohort of 10 formalin fixed paraffin embedded (FFPE) FTC tissues (4 with and 6 without PPFP). RNA from fresh frozen tissues and FFPE blocks was extracted using MiRVana and Recover All (Ambion) kits, respectively.

### In Vivo Xenograft Tumor Formation Assays

Stable cells (10⁷) were resuspended in 100 μL of growth media and injected subcutaneously into both flanks of 5 week-old athymic nude mice (Fox1^{nu/nu}, Harlan, Indianapolis, Ind.). At least three mice were injected per group. Tumor size was determined weekly by measuring tumor length (L), width (W), and height (H) using vernier calipers (Vₜᵤₘₒᵣ=0.5236 LWH).

### Tumor Histology and Immunohistochemistry

Four tumors per group were formalin-fixed, paraffin-embedded, and individual sections stained with CD-31 (sc-1506-R, Santa Cruz, 1:200 dilution) specific antibody for 30-60 minutes and counterstained with hematoxylin. Antigen retrieval was done in EDTA at 37° C. for 30 minutes after deparaffinization. Sections were blocked with peroxidase for 5 minutes and protein for 5 minutes. Antibody staining was visualized using Dako Envision plus System for 15 minutes followed by DAB chromogen for 10 minutes. Separate tumor sections were stained with hematoxylin and eosin to visualize tumor histology.

Stained slides were scanned using the Hamamatsu NanoZoomer (Bacus Laboratories, Inc., Chicago, Ill.). Digital images composed of multiple tiles (4096×64 pixel) were captured at 20× magnification. Image analysis was conducted as described elsewhere (Monahan et al., Endocrinology 150:4386-4394 (2009)). For each tumor section, the relative signal intensity from 10 randomly chosen fields (205×300 pixel) was quantified by histogram analysis (Adobe Photoshop). Data was expressed as signal intensity (signal pixel count divided by total image pixel count).

### Statistics

The statistical significance of difference between control and PPFP groups was determined by student's two-tailed t test, and p<0.05 was considered significant.

### Results

In trying to elucidate PPFP function in FTC tumorigenesis, it was demonstrated that the presence of PPFP in human tumors is associated with reduced CD31 staining, indicative of decreased neovascularization (Reddi et al., Expression of the PAX8/PPARγ Fusion Protein Is Associated with Decreased Neovascularization In Vivo: Impact on Tumorigenesis and Disease Prognosis, Genes and Cancer, in press (2010), in correlation with a study of a cohort of 54 FTC patients, wherein the 31 tumors that expressed PPFP were seen to exhibit favorable prognostic indicators, including better tumor differentiation, and lower risk of metastases (Sahin et al., J. Clin. Endocrinol. Metab., 90:463-468 (2005)). Meta-analysis of FTC cases evaluated in the literature (Table 1) revealed that 68% of FTC expressing PPFP are minimally invasive (χ²=6.86, p=0.008).

TABLE 1

| | | FTC | | PPFP | | PPFP | | |
|---|---|---|---|---|---|---|---|---|
| | No. of | No | | negative | | positive | | |
| Study | FTC | PPFP | PPFP | MI | WI | MI | WI | Study Reference |
| 1 | 15 | 7 | 8 | 6 | 1 | 4 | 4 | Nikiforova et al., Amer. J. Surg. |
| | | | | | | | | Pathol., 26: 1016-1023 (2002). |
| 2 | 9 | 4 | 5 | 4 | 0 | 5 | 0 | Marques et al., J. Clin Endocrinol. |
| | | | | | | | | Metab., 87: 3947-3953 (2002). |
| 3 | 33 | 16 | 12 | 7 | 9 | 1 | 11 | Nikiforova et al., J. Clin Endocrinol. |
| | | | | | | | | Metab., 88: 2318-2326 (2003). |
| 4 | 42 | 31 | 11 | 19 | 12 | 2 | 9 | French et al., Amer. J. Pathol., |
| | | | | | | | | 162: 1053-1060 (2003). |
| 5 | 17 | 11 | 6 | 7 | 4 | 5 | 1 | Cheung et al., J. Clin. Endocrinol. |
| | | | | | | | | Metab., 88: 354-357 (2003). |
| 4 | 34 | 24 | 10 | 20 | 4 | 8 | 2 | Dwight et al., J. Clin. Endocrin. |
| | | | | | | | | Metab., 88: 4440 (2003). |
| 5 | 21 | 17 | 4 | 5 | 12 | 1 | 3 | Lacroix et al., Eur. J. Endocrinol., |
| | | | | | | | | 151: 367-374 (2004). |
| 6 | 34 | 16 | 18 | 10 | 6 | 16 | 2 | Marques et al., Brit. J. Cancer, 91: 732-738 |
| | | | | | | | | (2004). |
| 7 | 54 | 23 | 31 | 0 | 23 | 28 | 3 | Sahin et al., J. Clin. Endocrinol. |
| | | | | | | | | Metab., 90: 463-468 (2005). |
| 8 | 27* | 12 | 10 | 7 | 5 | 6 | 4 | Castro et al., J. Clin. Endocrin. Metab., |
| | | | | | | | | 91: 213 (2006). |
| 9 | 17 | 10 | 7 | 4 | 6 | 7 | 0 | Banito et al., Clin. Endo., 67: 706-711 |
| | | | | | | | | (2007) |
| 10 | 12** | 8 | 4 | | | | | Nakabashi et al., Clin. Endo., 61: 280 |
| | | | | | | | | (2004) |
| Total | = 298 | = 176 | = 122 | = 89 | = 82 | = 83 | = 39 | |
| | | | | 50.5% | 46.5% | 68% | 32% | |

*PPFP Status not determined in 5 samples, all were classified as minimally invasive
**Break down of sample pathology not given, since there was no difference observed between the FTC ± PPFP
χ² 7.51, p = 0.006 uncorrected, and 6.86 Yates-corrected p = 0.008.

Follicular Thyroid Carcinoma Expressing PPFP are Associated With Increased miR-122 Expression

To identify the mechanism of anti-angiogenic function of PPFP in vivo, a cohort of 12 fresh frozen FTC tissues (6 each with and without PPFP), 10 folecular adenoma (4 expression PPFP and 6 without PPFP) and 7 normal thyroid tissues as controls were profiled using microRNA (miR) arrays. There were 139, 88, and 130 miRs, respectively, that distinguished (p<0.05, 2-fold change) FTC from normal, FTC from follicular adenoma (FA), and FTC with PPFP (FTC+PPFP) from FTC without PPFP (FTC−PPFP) (Table 2).

TABLE 2

| | miRNAs differentially regulated | | | |
|---|---|---|---|---|
| | (p < 0.05) & a 2-fold change | | | |
| | | Mean ± | Mean ± | Mean ± |
| Groups Compared | p < 0.05* | 1SD^{a} | 2SD^{a} | 3SD^{a} |
| FTC (12) vs Normal (7) | 139 | 74 | 26 | 9 |
| FA (10) vs FTC (12) | 88 | 34 | 5 | 1 |
| FTC-PPFP (6) vs FTC + | 130 | 73 | 26 | 8 |
| PPFP (6) | | | | |

^{a}Fold-change exceeded mean difference by 1, 2, or 3 standard deviaitions (SD).

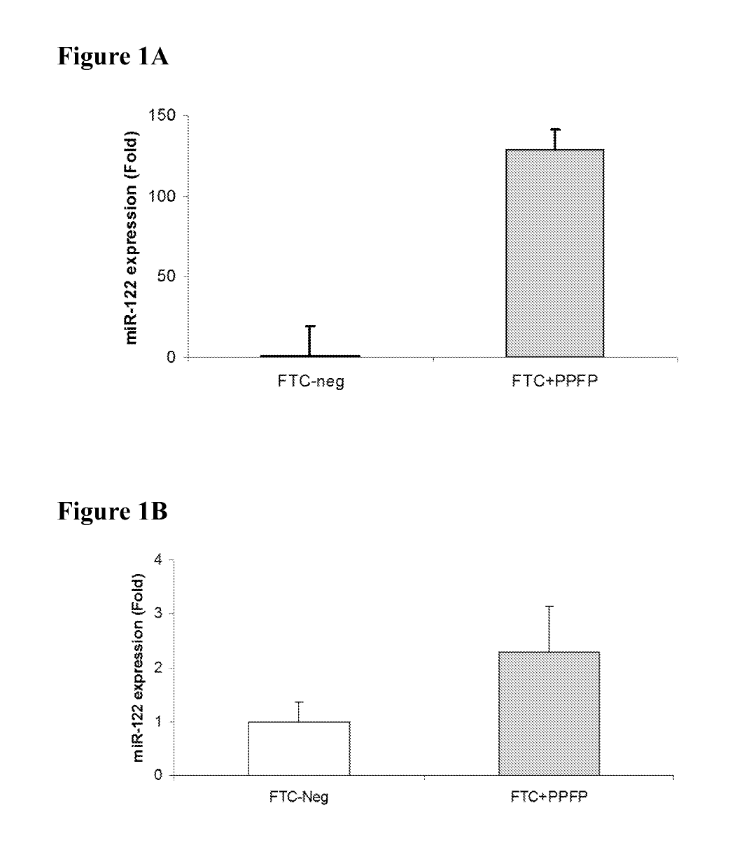

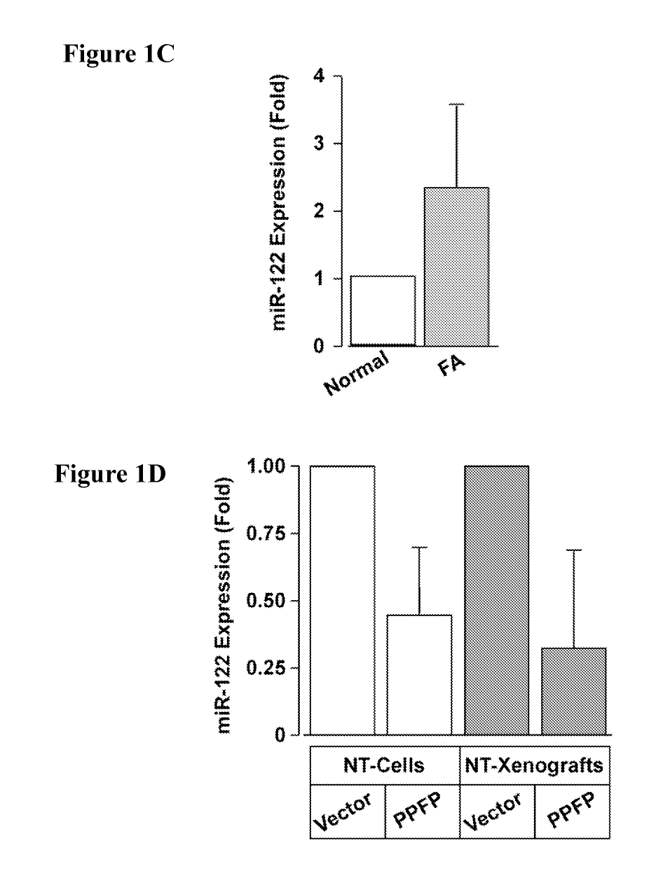

The initial studies focused on a set of 9 and 8 miRs that were differentially regulated at a fold difference of three standard deviations or higher in all FTC and FTC(+PPFP) compared to normal and FTC(−PPFP) respectively, Table 3. In both groups, miR-122 demonstrated a remarkably striking increase of 8.93- and 9.24-fold (Table 3) compared to normal thyroid and FA, respectively. More importantly, miR-122 was also able to distinguish a subset of FTC expressing PPFP (16.83-fold, p<0.001) from FTC that did not (Table 3), suggesting that it may be a PPFP-associated miR. Quantitative PCR (qPCR) of miR-122 expression in the same set of fresh frozen tumors demonstrated a 128-fold (p<0.05) increase of miR-122 in FTC(+PPFP) (FIG. 1A) compared to FTC−PPFP. These results were also validated in another independent cohort of 10 formalin fixed paraffin embedded FTC tissues (6 without and 4 with PPFP), which exhibited a 2.28-fold increase (p<0.05) in miR-122 in FTC+PPFP (FIG. 1B), but not in benign follicular adenomas (FIG. 1C) nor an adenoma model, consisting of immortalized thyrocytes (Nthy-ori 3-1 [NT] cells) constitutively expressing PPFP (FIG. 1D). These data confirm the miR profiling results and demonstrate that PPFP expression in follicular carcinomas, but not adenomas, is associated with significant up-regulation of miR-122 expression.

TABLE 3

| | | | | FTC + PPFP v. | |
|---|---|---|---|---|---|
| FTC v. normal | | FTC v. FA | | FTC-PPFP | |
| (mean ± 3SD^{a}) | | (mean ± 2SD^{a}) | | (mean ± 3SD^{a}) | |
| miRNA | FC^{b} | miRNA | FC^{b} | miRNA | FC^{b} |
| hsa-miR-122 | 8.93 | hsa-miR-122 | 9.24 | hsa-miR-122 | 16.83 |
| hsa-miR-1248 | 3.34 | has-miR-221 | 3.15 | hsa-miR-375 | 4.91 |
| has-miR-375 | 2.77 | has-miR-375 | 2.76 | hsa-miR-187 | 3.27 |
| hsa-miR-144* | 0.41 | HS_29 | 0.34 | hsa-miR-542-3p | 0.34 |
| hsa-miR-923 | 0.31 | has-miR-642 | 0.33 | hsa-miR-183 | 0.31 |
| solexa-8048-104 | 0.29 | | | hsa-miR-339-5p | 0.30 |
| hsa-miR-31* | 0.28 | | | hsa-miR-450b-5p | 0.27 |
| hsa-miR-31 | 0.26 | | | hsa-miR-542-5p | 0.27 |

^{a}Mean difference was greater than 2 or 3 standard deviations (SDs).
^{b}Fold change (FC).

### Constitutive Expression of PPFP in FTC-Derived Cell Lines Results in Reduced Tumor Progression in a Xenograft Mouse Model

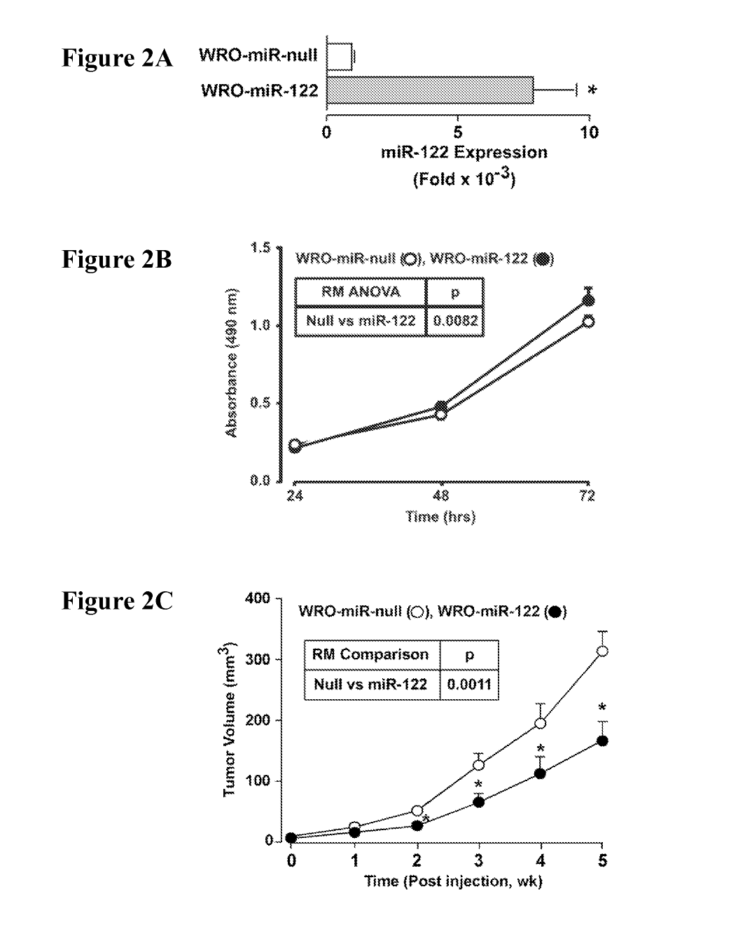

Stable transfection of a miR-122 precursor alone in WRO cells resulted in an 8,000-fold increase (p <0.005) in miR-122 expression as evaluated by quantitative RT-PCR (FIG. 2A). Overexpression of miR-122 in WRO cells increased in vitro growth slightly but significantly (11% increase at 72 hours, p<0.0082) (FIG. 2B). However, when these cells were transplanted into nude mice, tumor progression was inhibited by 1.8-fold (p<0.0011) (FIG. 2C), demonstrating that miR-122 can exert tumor suppressor activity in FTC-derived cells.

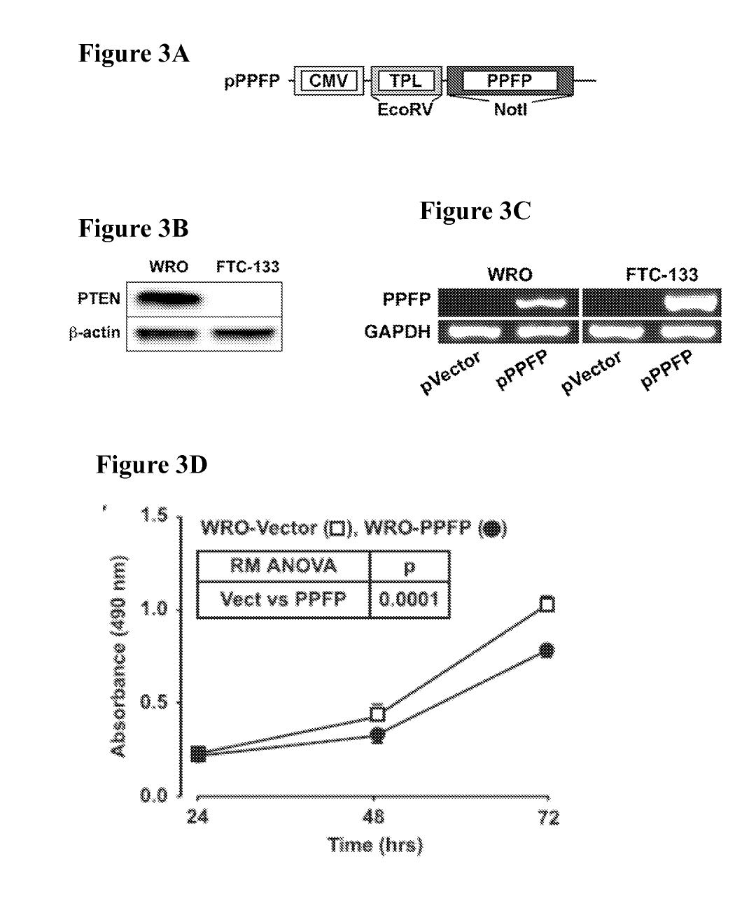

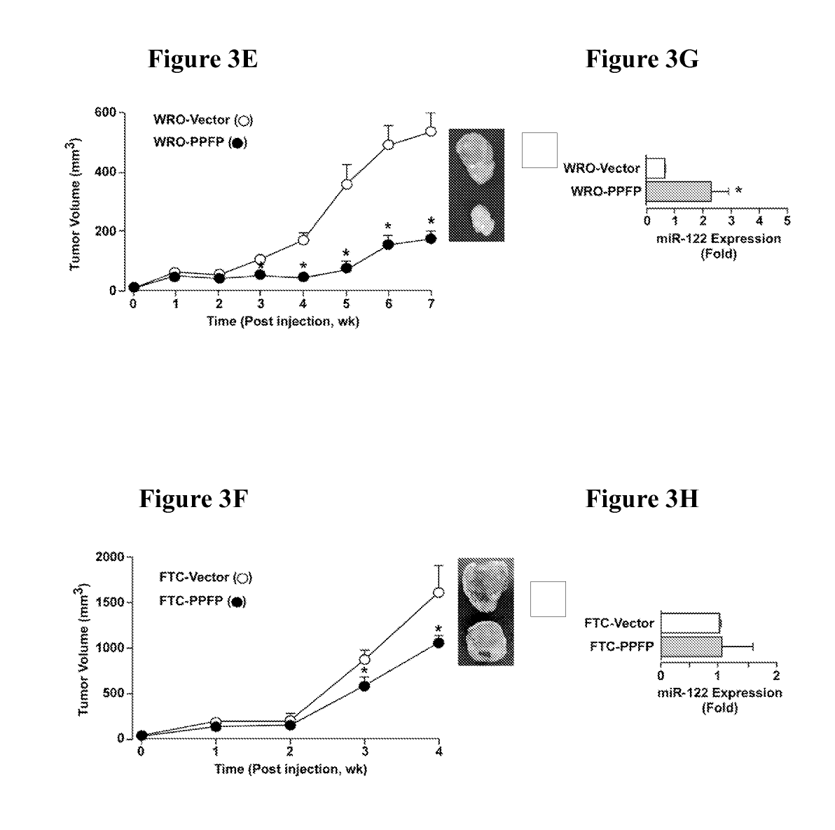

To understand the mechanism of PPFPs association with increased miR-122 expression in FTC, PPFP was constitutively expressed under the control of a CM V promoter (FIG. 3A) in two FTC-derived cell lines WRO (PTEN intact) and FTC-133 (PTEN-null). Expression of PTEN and PPFP was verified using western blotting (FIG. 3B and FIG. 3C, respectively). Expression of PPFP in WRO cells resulted in a 19% decrease (p<0.0001) in cell growth in vitro compared to WRO-vector cells (FIG. 3D). WRO-PPFP cells induced a 9-fold inhibition (p<0.00005) of xenograft tumor progression by 5 weeks (FIG. 3E), while FTC-133-PPFP induced a modest <2-fold inhibition (p<0.05) by 4 weeks (FIG. 3F) after tumor induction compared to vector only controls, indicating PTEN may help mediate the effects of PPFP. Quantitation of miR-122 levels demonstrated a 3-fold increase (p<0.05) only in the WRO-PPFP cells (FIGS. 3G and 3H). Additional samples were included and a comparison of miR-122 levels between WRO-PPFP cells and xenografts demonstrated an 8- and 40-fold increase (p<0.05) in the WRO-PPFP cells and xenografts, respectively (FIG. 3I), confirming that this WRO model recapitulates the properties seen in PPFP-expressing human FTC (Table 4).

TABLE 4
Relative miR-Expression (Fold Difference)
WRO models expressing PPFP

| Cell lines | Xenografts | FTC (+PPFP) |
|---|---|---|
| 8.0 (p < 0.05) | 40.0 (p < 0.054) | 16.8 (p < 0.001) |

Dominant Negative Inhibition of PPARγ is not Involved in PPFP Mediated Up-Regulation of miR-122

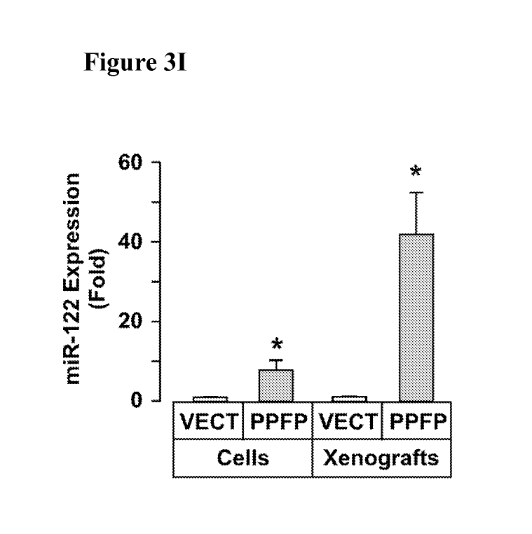

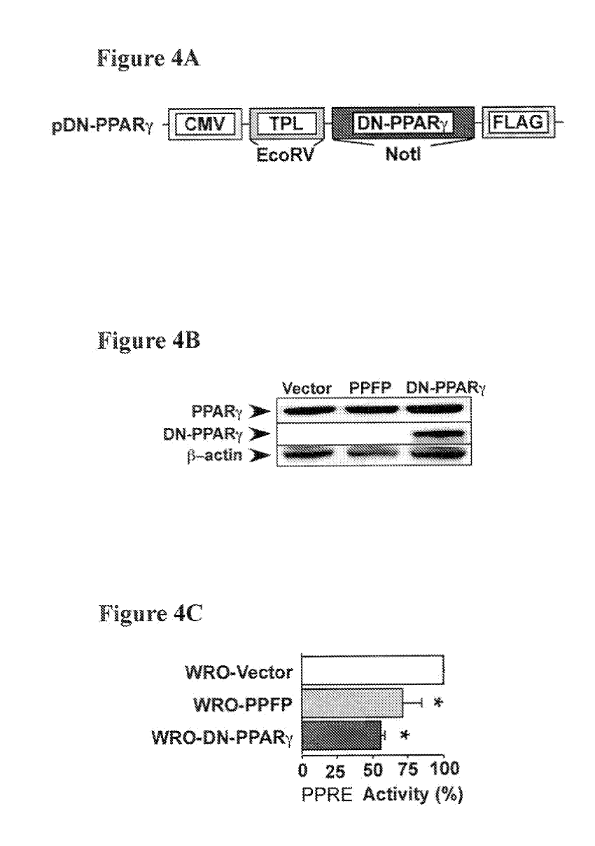

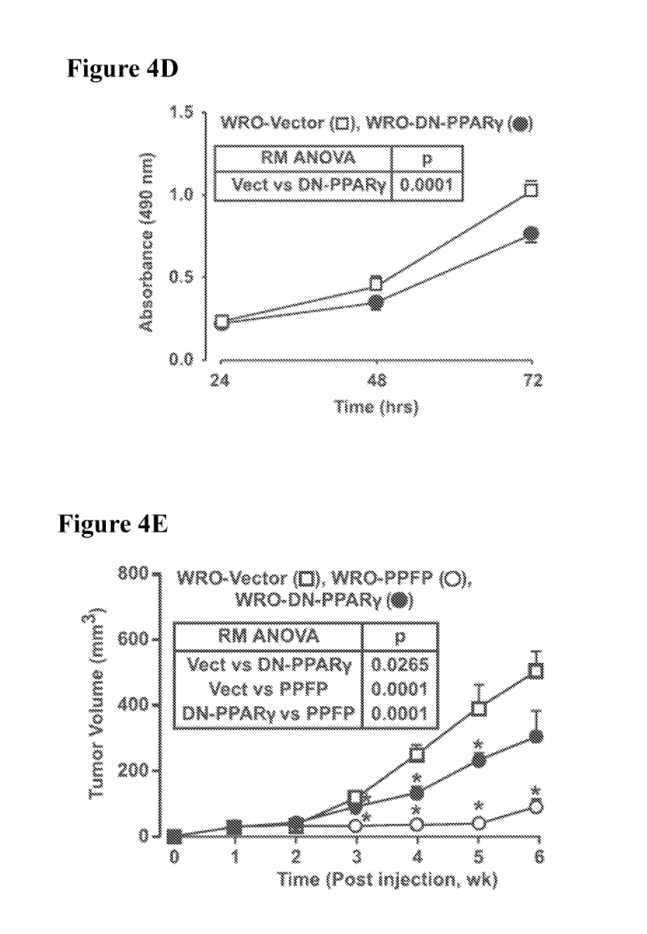

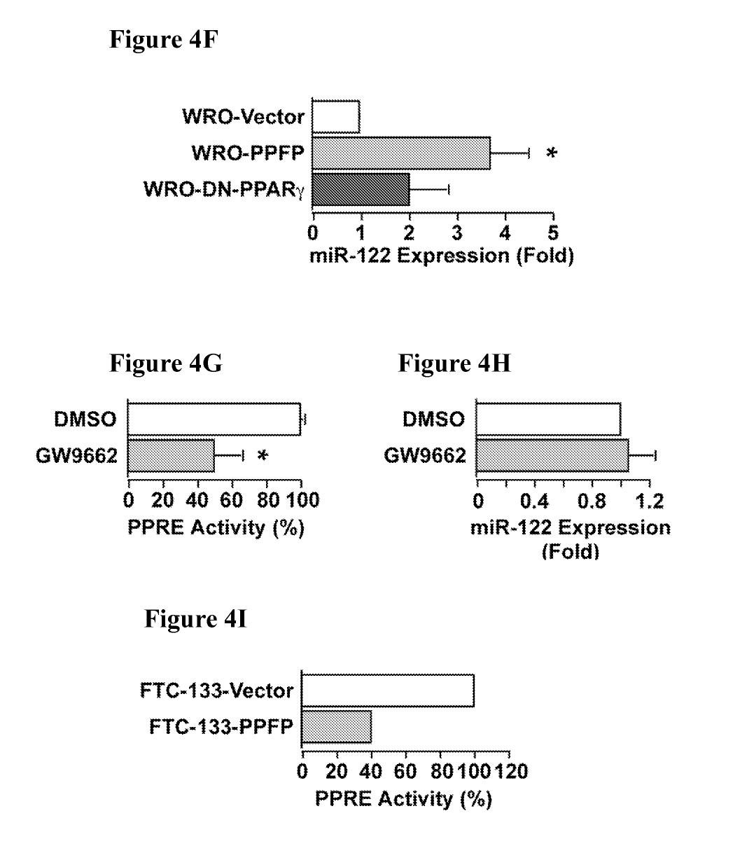

To understand whether the increase in miR-122 was mediated via the dominant negative (DN) inhibition of PPARγ of PPFP function, stable WRO cells expressing a DN-PPARγ mutant were generated using the construct shown in FIG. 4A (Reddi et al., Expression of the PAX8/PPARγ Fusion Protein Is Associated with Decreased Neovascularization In Vivo: Impact on Tumorigenesis and Disease Prognosis, Genes and Cancer, in press (2010)). Expression of the DN-PPARγ construct was confirmed by Western blot (FIG. 4B). Reporter assays confirmed that PPARγ activity, measured by activation of the PPARγ response element (PPRE), was significantly inhibited in both WRO-PPFP (29%) and WRO-DN-PPARγ (44%) cells (FIG. 4C), demonstrating that PPFP retains its dominant negative PPARγ function in the WRO-PPFP cells and that DN-PPARγ is expressed in a functional manner in WRO-DN-PPARγ cells. Expression of the DN-PPARγ mutant in WRO cells inhibited in vitro cell growth by 29% at 72 hours (p<0.0001) (FIG. 4D). Although this inhibition appeared to be somewhat greater than that of PPFP (19%), there was no significant difference as assessed by repeated-measures ANOVA. Evaluation of the tumorigenic potential in xenograft studies, however, revealed that the DN-PPARγ mutant was not as effective an inhibitor of tumorigenesis as PPFP, in that it exhibited only a modest inhibition of 3-fold (*p<0.05) compared to the vector control cells (FIG. 4E). Quantitation of miR-122 levels were not significantly increased when PPARγ was inhibited with a DN-PPARγ mutant (FIG. 4F), suggesting that up-regulation of miR-122 by PPFP was not mediated by dominant negative inhibition of PPARγ. Treatment of WRO-vector cells with GW9662, a potent antagonist of PPARγ confirmed these observations since there was a significant decrease in PPRE activity that was not accompanied by increased miR-122 expression (FIG. 4G and FIG. 4H). Also, reporter assays confirmed that activation of PPRE was significantly inhibited in FTC-133-PPFP (69%, p<0.05, FIG. 41), which could account for the modest inhibition of tumorigenesis. These results suggest that PPFP functions in vivo through multiple mechanisms, dominant negative inhibition of PPARγ and up-regulation of miR-122.

PTEN is Involved in PPFP Mediated Up-Regulation of miR-122

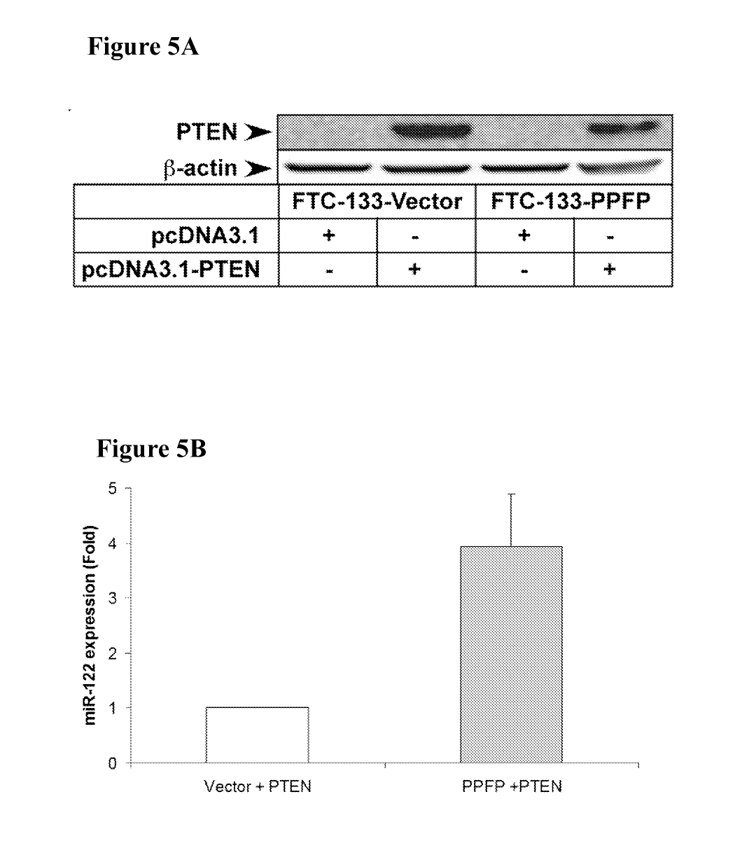

The results provided herein indicate that PPFP function involves two independent mechanisms: dominant negative inhibition of PPARγ and up-regulation of miR-122. Since expression of PPFP in the PTEN-null FTC-133 resulted in a modest inhibition of tumorigenesis similar to either expression of miR-122 alone or the DN-PPARγ mutant, together with the observation that FTC-133(+PPFP) cells did not increase miR-122 expression, PTEN may be involved in PPFP mediated miR-122 up-regulation. Transient expression of PTEN in the FTC-133(+PPFP) cells (FIG. 5A) dramatically increased miR-122 expression 77-fold (p<0.05) compared to the vector only cells (FIG. 5B), confirming that PTEN is involved in PPFP mediated increase of miR-122.

Upregulation of miR-122 in WRO Cells Reduces Expression of the Pro-Angiogenic Factor ADAM-17

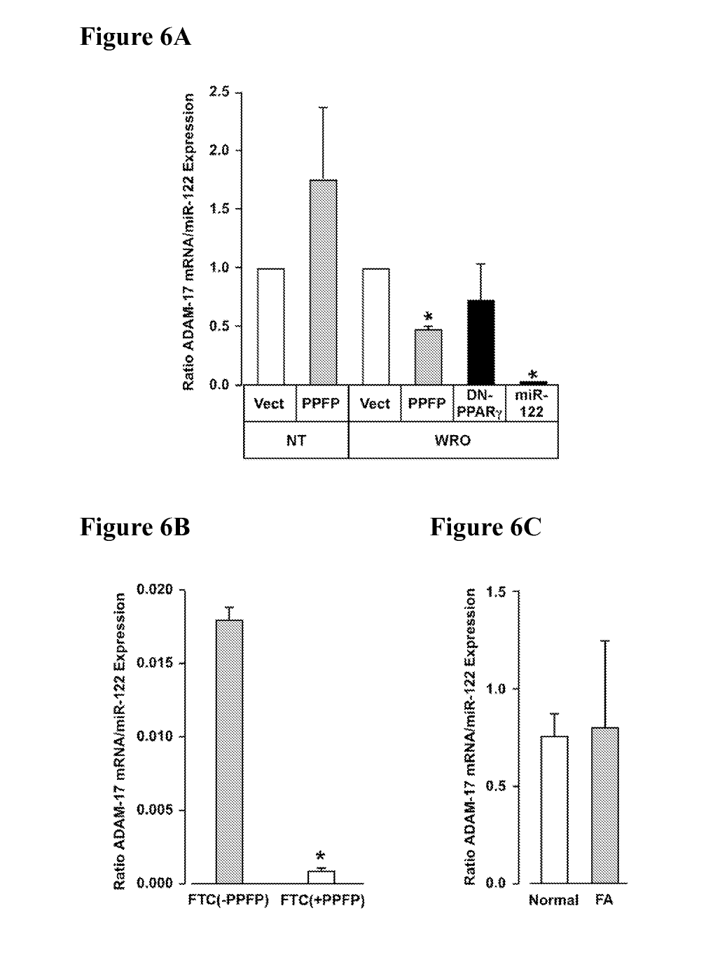

To ascertain whether PPFP-mediated up-regulation of miR-122 had functional consequences in our model systems, transcript levels of ADAM-17, a known downstream target of miR-122, were evaluated. Reduction of ADAM-17 mRNA expression was observed only in cell lines that display significant up-regulation of miR-122, including the WRO-miR-122 cells (100% reduction, p<0.0001) and WRO-PPFP cells (50% reduction, p<0.001), but not immortalized thyrocytes (N-Thy-ori cells) expressing PPFP (FIG. 6A). These data confirmed that the up-regulated miR-122 expression in these cells is functional. Evaluation of ADAM-17 expression in human tumors also demonstrated that ADAM-17 was reduced 20-fold (p<0.05) only in the follicular carcinomas that express PPFP (FIG. 6B), but not in the adenomas (FIG. 6C). These data indicate that PPFP-mediated up-regulation of miR-122 in our cell lines and human tumors exerts functional consequences on miR-122 downstream targets and suggests that PPFP-mediated miR-122 upregulation may act as a tumor growth modulator by inhibiting angiogenic pathways.

### PPFP-Mediated Inhibition of Neovascularization Partially Accounts for Tumor Suppressor Function

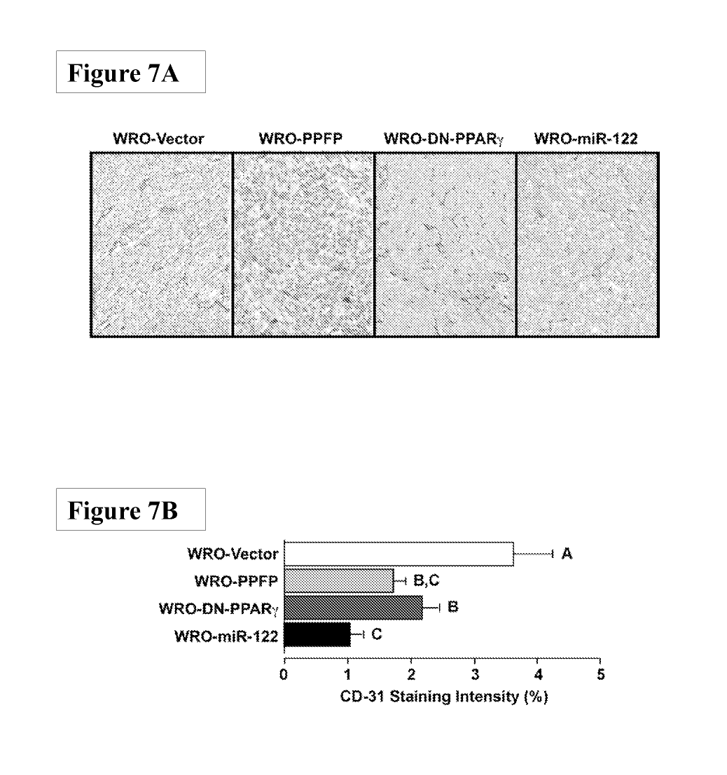

Given our previous observations that PPFP expression in human adenomas and carcinomas is associated with decreased neo-vascularization (24), the WRO-Vector, -PPFP, -DN-PPARγ and -miR-122 xenografts were evaluated for microvessel density (MVD) as evidenced by CD-31 staining (FIG. 7A). Quantitation of CD-31 staining demonstrated that MVD was significantly decreased 2.1-, 1.7- and 3.4-fold, respectively in the WRO-PPFP, -DN-PPARγ and -miR-122 xenografts compared to control xenografts, but they were not significantly different from each other (FIG. 6B).

The results provided herein also demonstrate that (1) miR-122 is over expressed in FTC as a whole and can be used as a marker to distinguish benign FA from malignant FTC, (2) increased expression of miR-122 in FTC can be responsible for the associated decreased angiogenesis observed in comparison to FA or normal thyroid tissue, and (3) expression of the PAX8/PPARγ rearrangement further enhances expression of miR-122 resulting in favorable prognosis for a specific subset of FTC. Thus, as described herein, miR-122 can be used as a diagnostic and prognostic marker for FTC.

### Example 2

### Expression of PAX8/PPARγ Fusion Protein Reduces Growth of Colon Cancer, Melanoma, and Anaplastic Thyroid Cancer Tumors

### Generation of Stable Cells

ARO, DRO, FRO, and KTC-3 cells (obtained from Dr. John A Copland, Mayo Clinic Jacksonville) were grown in RPMI-1640 medium (Invitrogen, Carlsbad, Calif.) supplemented with 10% FBS (Hyclone, Logan, Utah) and 1× penicillin-streptomycin (Invitrogen) at 37° C. in humidified conditions with 5% CO₂. PPFP constructs were generated as described elsewhere (Reddi et al., Expression of the PAX8/PPARγ Fusion Protein Is Associated with Decreased Neovascularization In Vivo: Impact on Tumorigenesis and Disease Prognosis, Genes and Cancer, (2010)).

To generate stable lines, cells were seeded at 1×10⁶ cells per 100 mm petri dish and transfected 24 hours later in multiples of six, with 10 μg each of vector or PPFP using LT-1 (Minus Bio, Madison, Wis.). After 72 hours, ARO-Vector, ARO-PPFP, DRO-Vector, DRO-PPFP, FRO-Vector, FRO-PPFP, KTC-3-Vector, and KTC-3-PPFP cells were obtained by selection in media containing 400 μg/mL of G418 (Invitrogen) with media changes every 72 hours. Pools of cells were harvested and analyzed for PPFP expression by reverse transcriptase PCR.

### In Vivo Xenograft Tumor Formation Assays

Stable cells (10⁷) were resuspended in 100 μL of growth media and injected subcutaneously into both flanks of 5 week-old athymic nude mice (Fox1^{nu/nu} Harlan, Indianapolis, Ind.). At least three mice were injected per group. Tumor size was determined weekly by measuring tumor length (L), width (W), and height (H) using vernier calipers (Vₜᵤₘₒᵣ=0.5236 LWH).

### Results

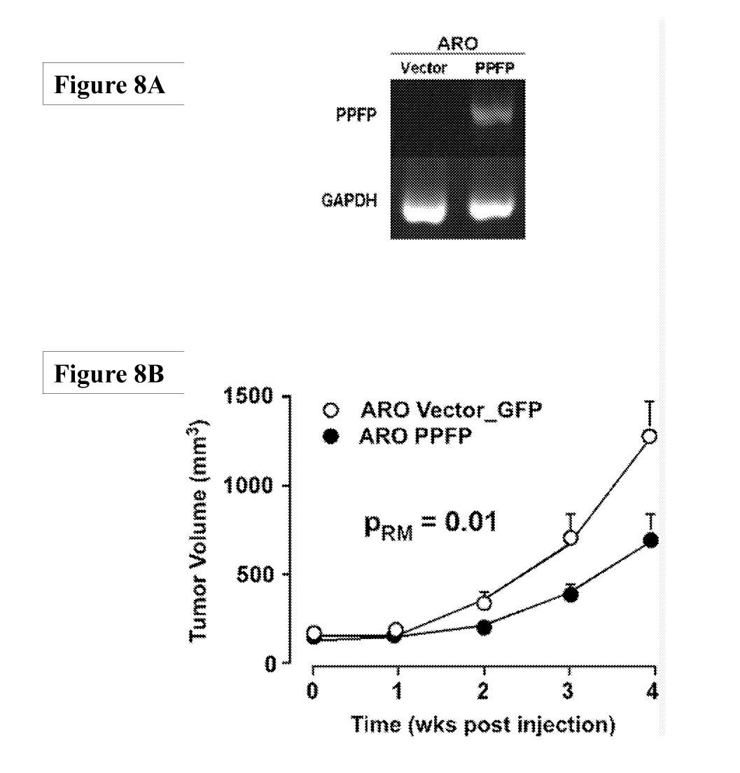

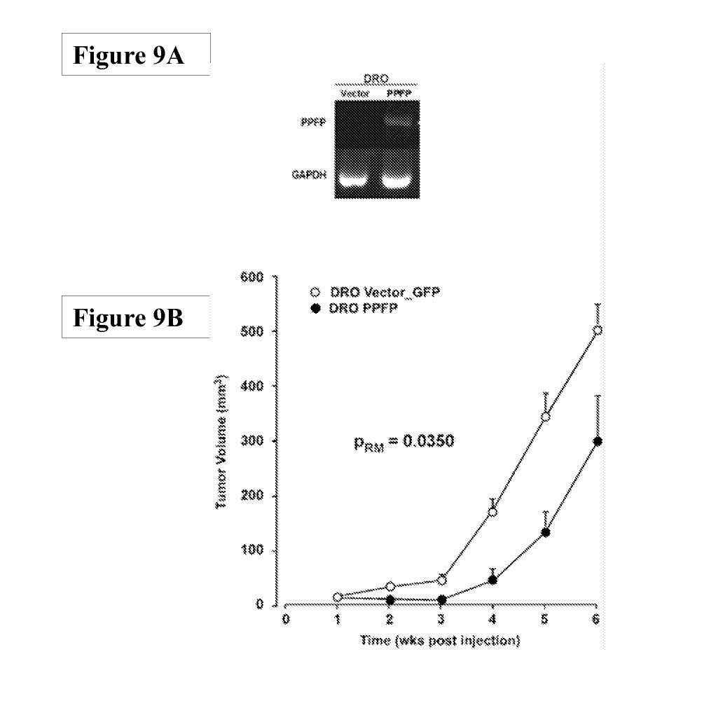

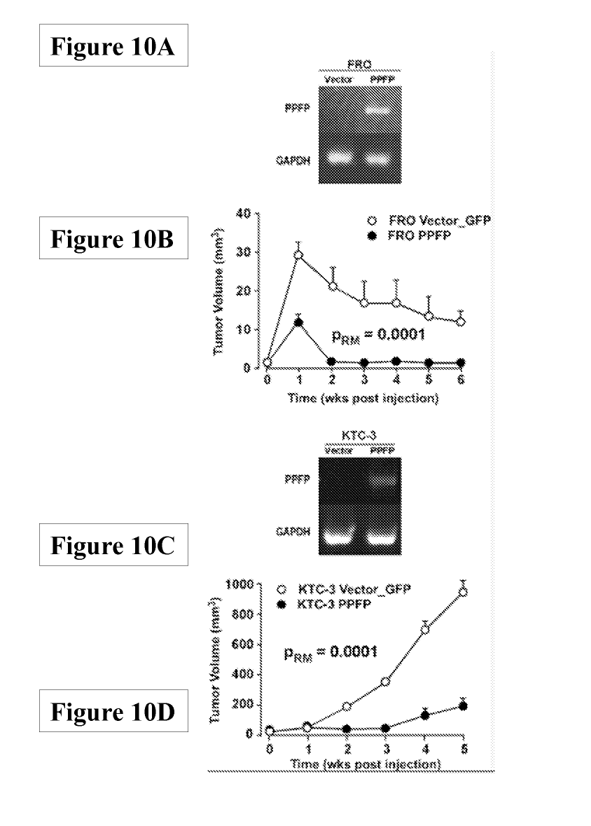

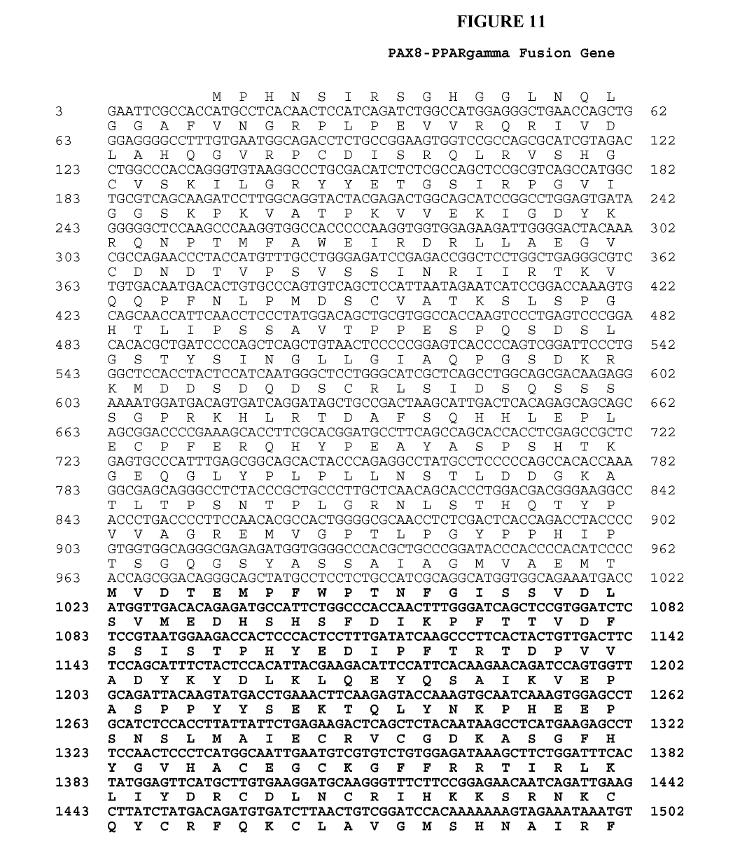

Expression of PPFP was verified by reverse transcriptase PCR in ARO, DRO, FRO, and KTC-3 cells (FIGS. 8A, 9A, 10A, and 10C, respectively). The colon cancer cells (ARO-PPFP) induced a 39.5% inhibition (p<0.01) of xenograft tumor progression by 4 weeks (FIG. 8B). Melanoma cells (DRO-PPFP) induced a 57.5% inhibition (p<0.035) of xenograft tumor progression by 6 weeks (FIG. 9B). Anaplastic thyroid cancer cells, FRO-PPFP and KTC-3-PPFP, induced a 80.1% in 5 weeks (p<0.0001 and 77.2% in 6 weeks (p<0.0001) inhibition, respectively of xenograft tumor progression (FIGS. 10B and 10D). WRO-PPFP cells induced a 72% inhibition (p<0.00005) of xenograft tumor progression by 5 weeks (FIG. 3E).

The results provided herein demonstrate that the expression of PPFP in cancer cells could be used as a novel potential therapeutic treatment for colon cancer, melanoma, and anaplastic thyroid cancer.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-29. (canceled)

30. A method for treating a mammal having cancer cells that lack expression of a PPFP polypeptide, wherein said method comprises administering, to said mammal, a nucleic acid comprising a nucleic acid sequence that encodes said PPFP polypeptide under conditions wherein said cancer cells express said PPFP polypeptide.

31. The method of claim 30, wherein said mammal is a human.

32. The method of claim 30, wherein said cancer cells are colon, melanoma, follicular thyroid cancer, or anaplastic thyroid cancer cells.

33. The method of claim 30, wherein said nucleic acid is administered to said mammal under conditions wherein expression of microRNA 122 (miR122) in said cancer cells is increased.

34. The method of claim 30, wherein the volume of a tumor comprising said cancer cells decreases after said administration.

35. The method of claim 34, wherein said decrease is a decrease of at least about 10 percent.

36. The method of claim 34, wherein said decrease is a decrease of at least about 25 percent.


SEQUENCE LISTING (4 sequences)

SEQ ID NO: 1 - DNA, 27 bp, Homo sapiens
cgcggatccgcattgactcacagagca

SEQ ID NO: 2 - DNA, 27 bp, Homo sapiens
ccggaattcgaagtcaacagtagtgaa

SEQ ID NO: 3 - protein, 810 aa, Homo sapiens
MPHNSIRSGHGGLNQLGGAFVNGRPLPEVVRQRIVLAHQGVRPCDISRQLRVSHGCVSKILGRYYETGSIRPGVIGGSKPKVATPKVVEKIGDYKRQNPTMFAWEIRDRLLAEGVCDNDTVPSVSSINRIIRTKVQQPFNLPMDSCVATKSLSPGHTLIPSSAVTPPESPQSDSLGSTYSINGLLGIAQPGSDKRKMDDSDQDSCRLSIDSQSSSSGPRKHLRTDAFSQHHLEPLECPFERQHYPEAYASPSHTKGEQGLYPLPLLNSTLDDGKATLTPSNTPLGRNLSTHQTYPVVAGREMVGPTLPGYPPHIPTSGQGSYASSAIAGMVAEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 4 - DNA, 2502 bp, Homo sapiens
gaattcgccaccatgcctcacaactccatcagatctggccatggagggctgaaccagctgggaggggcctttgtgaatggcagacctctgccggaagtggtccgccagcgcatcgtagacctggcccaccagggtgtaaggccctgcgacatctctcgccagctccgcgtcagccatggctgcgtcagcaagatccttggcaggtactacgagactggcagcatccggcctggagtgatagggggctccaagcccaaggtggccacccccaaggtggtggagaagattggggactacaaacgccagaaccctaccatgtttgcctgggagatccgagaccggctcctggctgagggcgtctgtgacaatgacactgtgcccagtgtcagctccattaatagaatcatccggaccaaagtgcagcaaccattcaacctccctatggacagctgcgtggccaccaagtccctgagtcccggacacacgctgatccccagctcagctgtaactcccccggagtcaccccagtcggattccctgggctccacctactccatcaatgggctcctgggcatcgctcagcctggcagcgacaagaggaaaatggatgacagtgatcaggatagctgccgactaagcattgactcacagagcagcagcagcggaccccgaaagcaccttcgcacggatgccttcagccagcaccacctcgagccgctcgagtgcccatttgagcggcagcactacccagaggcctatgcctcccccagccacaccaaaggcgagcagggcctctacccgctgcccttgctcaacagcaccctggacgacgggaaggccaccctgaccccttccaacacgccactggggcgcaacctctcgactcaccagacctaccccgtggtggcagggcgagagatggtggggcccacgctgcccggatacccaccccacatccccaccagcggacagggcagctatgcctcctctgccatcgcaggcatggtggcagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactaggtcgacgcgtaagccgaattctgcagatatccagcacagtggcggccgctcgag